(19) United States
(12) Patent Application Publication
(10) Pub. No.: US 2021/0259588 A1
(43) Pub. Date: Aug. 26, 2021

(54) APPARATUS, SYSTEM AND METHOD TO PROVIDE A PLATFORM TO OBSERVE BODILY FLUID CHARACTERISTICS FROM AN INTEGRATED SENSOR STRIP

(71) Applicant: JABIL INC., St. Petersburg, FL (US)

(72) Inventors: Girish WABLE, St. Petersburg, FL (US); Yusuf BHAGAT, St. Petersburg, FL (US); Thomas HARTRANFT, St. Petersburg, FL (US); David Michael ANTHUS, St. Petersburg, FL (US)

(73) Assignee: Jabil Inc., St. Petersburg, FL (US)

(21) Appl. No.: 17/047,055

(22) PCT Filed: Apr. 11, 2019
(86) PCT No.: PCT/US19/27070

§ 371 Date: Oct. 12, 2020

Related U.S. Application Data
(60) Provisional application No. 62/656,398, filed on Apr. 12, 2018.

(51) Int. Cl. A61B 5/145; A61B 5/00

(52) U.S. Cl. CPC A61B 5/14517; A61B 5/6898; A61B 2562/0295; A61B 5/14539; A61B 5/0059; A61B 5/6824

(57) ABSTRACT

The disclosure is and includes at least an apparatus, system and method for correlating bodily fluids to health aspects. The apparatus, system and method includes a slug comprising a passive chemical sensor, a receiving channel for wicking the bodily fluids from a body to the passive chemical sensor, and an interface for interfacing the passive chemical sensor to a mobile device; at least one indicator associated with the passive chemical sensor, wherein the at least one indicator changes based on features of the wickable bodily fluids; and at least one computing memory device associated with the mobile device comprising at least comparative lookup table of an application, wherein the at least one indicator is compared to the comparative lookup table to produce a user display on the application of the health aspects indicated by the at least one indicator.

### CROSS-REFERENCE TO RELATED APPLICATION

This application is a national stage application of International Patent Application No. PCT/US2019/027070, filed Apr. 11, 2019, entitled: APPARATUS, SYSTEM AND METHOD TO PROVIDE A PLATFORM TO OBSERVE BODILY FLUID CHARACTERISTICS FROM AN INTEGRATED SENSOR STRIP, which claims the benefit of priority to U.S. Provisional Application No. 62/656,398, filed Apr. 12, 2018, entitled APPARATUS, SYSTEM AND METHOD TO PROVIDE A PLATFORM TO OBSERVE BODILY FLUID CHARACTERISTICS FROM AN INTEGRATED SENSOR STRIP, the entireties of which is incorporated herein by reference as if set forth in its entireties.

### BACKGROUND

### Field of the Disclosure

The disclosure relates generally to sensors and, more particularly, to an apparatus, system and method to provide a platform to observe bodily fluid characteristics from an integrated sensor strip.

### Background of the Disclosure

Exercise is a significant component of healthy living. The fitness industry, and particularly technologies related to improving the impact of exercise and monitoring exercise results, is a vital aspect upon which millions depend in order to improve their health. At present, the monitoring aspects of the fitness industry upon which these millions depend comprise, for the most part, activity monitors, i.e., step trackers. This is the case notwithstanding that simple activity tracking does not have an exceedingly high correlation to healthy living and exercise impact.

On the other hand, bodily fluids do have a high correlation to health. However, bodily fluids are difficult to monitor and extract information from, at least without performing invasive bodily tasks at home, sending away samples for testing, or suffering the inconvenience of visiting a doctor or a hospital.

Although numerous bodily fluids provide chemical indications of the impact of exercise (among many other health-related indications provided by bodily fluids), the sweat of a user is one of the most, if not the most, significant indicator of the impact of exercise. Similarly, saliva is a valuable indicator of other aspects of health, and other bodily fluids, such as tears, urine, and so on are significant indicators of particular aspects of healthy living.

The body produces sweat, such as during exercise or other strenuous or stressful periods, through the sweat glands. The sweat glands are comprised of two principal components, namely a tubular coiled area in which sweat is produced (at near iso-osmotic range, with a pH of approximately 7.0), and a duct through which sweat reaches the surface of the skin (in the hypo-osmotic range, with a pH of substantially less than 7.0). Perspiration, or sweating, results due to the reabsorption process within the sweat duct. For example, during exercise, the sweat rate excreted increases in order to regulate body temperature, and therefore the time for reabsorption of sweat decreases. This results in an increase in the pH of sweat, making it less acidotic and more alkaline over time.

Numerous regions of the body generate the most significant amount of sweat, such as the forehead, the chest, and the lower back, although nearly all areas of the body produce sweat under strenuous circumstances. Table 1, below, provides a list of the various electrolytes, metabolites, small molecules, and proteins typically found in sweat. Table 2, also below, focuses on the target electrolytes which may be sensed during sweating, as these target electrolytes may correlate most highly to aspects of health. Table 2 illustrates the relationship of the rate of sweating and the relative concentrations of the target electrolytes.

TABLE 1

| Electrolytes | Metabolites | Small Molecules | Proteins |
|---|---|---|---|
| Sodium | Lactate | Amino acids | Interleukins |
| Chloride | Creatinine | DHEA | Tumor necrosis factor |
| Potassium | Glucose | Cortisol | Neuropeptides |
| Calcium | Uric acid | | |

TABLE 2

| | | Concentration range |
|---|---|---|
| | Sweat rate dependent (active)/ | (mM) in sweat @ |
| Electrolyte | independent (passive) | surface |
| Sodium | Dependent | 10-100 |
| Chloride | Dependent | 10-100 |
| Potassium | Independent | 4-24 |
| Ammonium | Independent | 0.5-8 |

The main function of sweating is thermal regulation of the body. Hence, real-time analysis of the sweat concentrations above, and in addition, the balances of numerous other elements of sweat, may be a valuable tool in the diagnoses and analyses of various health aspects. For example, sweat may provide information in relation to: cystic fibrosis diagnosis (which is based on sodium and chloride concentration levels); the detection of metabolic alkalosis (which is the buildup of serum bicarbonate) during exercise due to the close associations between sweat, pH, and electrolyte concentrations (which are indicative of muscle fatigue markers); hyperhidrosis (which is excessive sweating), wherein electrolytes may be depleted by severe exertion; and electrolyte balance and an extent of rehydration, which are evidenced by the varying concentration of electrolytes between different individuals, and which may lead to personalized rehydration strategies.

The pH range of sweat may range from 4.0 to 8.0. However, measurement of a simple pH range may provide little in the way of true indication of analytics of sweat, and hence current sweat collection and analysis methods suffer from severe limitations as bio-marker monitoring systems. For example, these limitations on existing sweat collection and analysis methods include significant limitations on the weight measurement methodology for sweat collection analytics. This analytic method, in which changes in body weight prior to- and post-exercise are measured, is impractical for routine assessment of sweat and individuals, even during athletic performance, because of the poor correlation between weight variation and the sweat output indications.

Similarly, Minor's method is a method in which the skin is covered with a starch iodine powder, which enables a colorimetric detection scheme in which purple dots are visible in concert with the appearance of sweat droplets. This method suffers from the significant drawback that simple indications of the numbers or amount of sweat droplets bears little in the way of successful data indicators.

Wash down techniques involve exercise within plastic enclosures to allow for the collection of sweat, and after exercise the body is washed down inside the enclosure with deionized water. Again, this method suffers from numerous drawbacks, including variations between exercisers, poor collection results, and the insignificance of correlation to actual exercise results.

In a power film assessment, patches are attached to the skin, such as with a wound-type dressing, and sweat is then aspirated throughout a predetermined trial. Thereby, sweat rates may be assessed a grade metrically, and bio impedance (which is a complex analysis methodology that necessitates the placement of electrodes), as continuously measurement by complex circuitry, may be used to assess overall hydration.

Of note, and with respect to each of the foregoing examples of existing bodily fluid collection and analysis methods, the greatest challenge for sensing bodily fluids in real time remains, in the known art, obtaining and processing uncontaminated samples. Therefore, the need exists for an apparatus, system and method of sensing bodily fluids without contamination and of correlating those measurements to health aspects.

### SUMMARY

The disclosure is and includes at least an apparatus, system and method for correlating bodily fluids to health aspects. The apparatus, system and method includes a slug comprising a passive chemical sensor, a receiving channel for wicking the bodily fluids from a body to the passive chemical sensor, and an interface for interfacing the passive chemical sensor to a mobile device; at least one indicator associated with the passive chemical sensor, wherein the at least one indicator changes based on features of the wicked bodily fluids; and at least one computing memory device associated with the mobile device comprising at least comparative lookup table of an application, wherein the at least one indicator is compared to the comparative lookup table to produce a user display on the application of the health aspects indicated by the at least one indicator.

Thus, the disclosure provides an apparatus, system and method of sensing bodily fluids without contamination and of correlating those measurements to health aspects.

### BRIEF DESCRIPTION OF THE DRAWINGS

The disclosure is illustrated by way of example and not limitation in the accompanying drawings, in which like references indicate similar elements, and in which:

FIG. 1 illustrates a system diagram according to embodiments;

FIG. 2 illustrates a diagrammatic representation according to embodiments;

FIG. 3 illustrates a schematic block diagram according to embodiments; and

FIG. 4 illustrates a flow diagram according to embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

Computer-implemented platforms, engines, systems and methods of use are disclosed herein that provide networked access to a plurality of types of digital content, including but not limited to video, image, text, audio, metadata, algorithms, interactive and document content, and that track, deliver, manipulate, transform and report the accessed content. Described embodiments of these platforms, engines, systems and methods are intended to be exemplary and not limiting. As such, it is contemplated that the herein described systems and methods may be adapted to provide many types of server and cloud-based valuations, interactions, data exchanges, and the like, and may be extended to provide enhancements and/or additions to the exemplary platforms, engines, systems and methods described. The disclosure is thus intended to include all such extensions.

Furthermore, it will be understood that the terms “module” or “engine”, as used herein does not limit the functionality to particular physical modules, but may include any number of tangibly-embodied software and/or hardware components having a transformative effect on at least a portion of a system. In general, a computer program product in accordance with one embodiment comprises a tangible computer usable medium (e.g., standard RAM, an optical disc, a USB drive, or the like) having computer-readable program code embodied therein, wherein the computer-readable program code is adapted to be executed by a processor (working in connection with an operating system) to implement one or more functions and methods as described below. In this regard, the program code may be implemented in any desired language, and may be implemented as machine code, assembly code, byte code, interpretable source code or the like (e.g., via C, C++, C#, Java, Actionscript, Objective-C, Javascript, CSS, XML, etc.).

The embodiments provide an apparatus, system and method for sensing bodily fluids and for correlating those measurements to health aspects. More particularly, the disclosure may provide sensor strips that may enable the measurement of chemical composition of bodily fluids. These sensor strips may use a mobile lighting and imaging platform, such as may be present in a mobile phone or in a dedicated mobile device.

In embodiments, a platform may be provided to house sensor strips that wick sweat and/or other bodily fluids from the skin and/or body and channel it to the light and imaging sensor platform. The sensor strips may be placed, for example, in a wrist or armband in which physical association with a mobile phone's imaging device may occur. Alternatively, the sensor strips may be taken from a sweat collection point, such as a wrist or armband, and affirmatively placed in association with a slot, such as in a mobile device protective cover or case, which allows for measurement by the imager of the mobile device of sweat concentrations.

Yet further, the embodiments may include a dedicated wearable, such as wrist or arm-wearable, mobile device that includes a sensor strip, lighting and an imager. The dedicated mobile device may be capable of relaying results, such as from a device memory, over a wireless connection or a wired connection, to an analytic software platform, such as may reside on a nearby user's mobile device.

As referenced, the embodiments leverage a light source, such as may be available on a typical mobile device. The light source may create a lighted spot on an ambient sensor having appropriate reflectance. That is, the light source may suitably light the sensor to allow for imaging of the sensor status by a nearby imager. Accordingly, sensing may include the use of the front or rear facing camera module (i.e., light/imager combination) on a mobile phone to view an adjacent sensor strip, such that the processing power and graphical user interface capabilities of the mobile device may be leveraged to readily relay the status of the sensed bodily fluids to a user. For example, the mobile device may employ an app or similar software environment in order to analyze and relay the bodily fluid data gained.

The disclosure may leverage existing low cost disposable chemical sensor strips, such as may sense pH or ammonia, by way of non-limiting example, via, for example, color changes of said sensor strips. Of course, other secondary sensors or sensor types may be associated with the embodiments, such as hydration sensors or heart monitoring sensors, by way of non-limiting example, in order to provide a fuller picture of aspects of the user's health aspects.

Further and as mentioned, existing and well known exercise equipment, such as an armband having pocket into which a smart phone is placed, may be leveraged in the embodiments to enable analysis of particular chemical aspects. For example, the embodiments may allow for placement of a sensor strip in association with the pocket of the armband, such that a phone placed into the pocket may actuate and have access to the sensor strip. Such features may be enhanced by add-on sensors or further integration of additional sensors, such as heart rate sensing, in order to provide more holistic biomarker sensing.

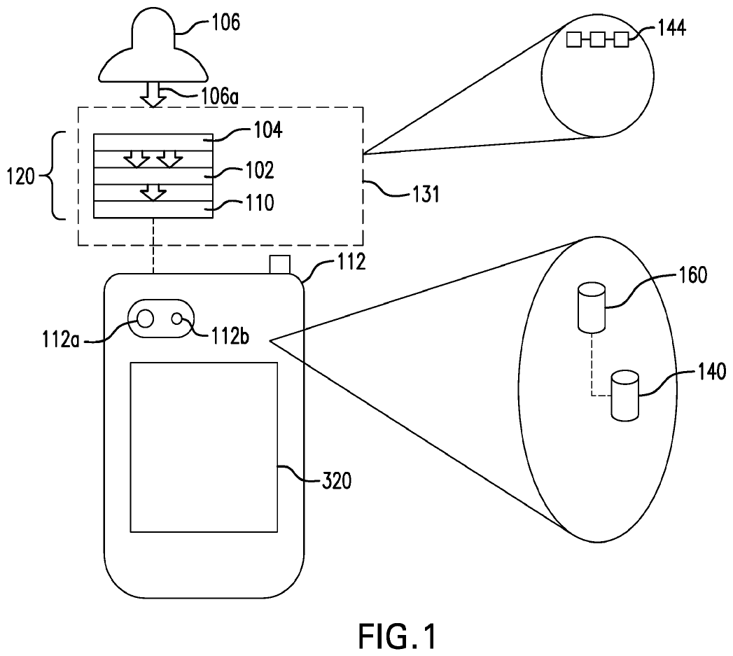

More particularly, and as illustrated in the example of FIG. 1, a sensing strip 102, such as a pH or ammonia sensing strip, may be housed in or in association with, for example, a receiving channel 104, such that the strip 102 may be brought into close contact with the user 106, such as with the user's skin or mouth 106. The strip 102 may be subjected to receipt of the user's bodily fluid 106a, such as the user's sweat, such as by a wicking of the bodily fluid 106a away from the skin to allow for absorption by the strip 102. The wicking to the sensor strip 102 may occur using a physical device interface, such as a wicking channel 104, which may be formed of any substance known to the skilled artisan to be suitable to wick bodily fluids towards the sensor strip 102.

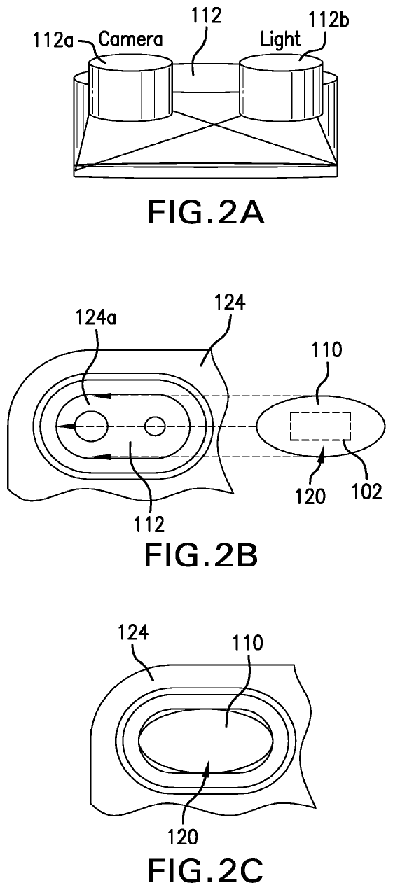

A device interface 110 may then be associated with a mobile device 112, such as a smart phone, in order to allow for interfacing with the imaging system 112a and light source 112b of the mobile device 112. For example, the mobile device 112 may fit into a vehicle 131, such as an armband, which, when the mobile device 112 is situated in the armband, places the imaging system 112a and lighting 112b of the mobile device 112 into physical association with the interface 110 (which may be integral with the armband or similar vehicle). Likewise and as additionally illustrated with respect to FIGS. 2A, 2B and 2C, the interface 110, such as the wicking channel, may be present in a slug 120 which may form part of a vehicle into which the mobile device 112 is placed, or which may be fittedly inserted into a slot of, or otherwise interfaced with, a smart phone 112 or a smart phone case 124, such as into a slot 124a thereof, in order to allow for direct interface with the camera and light source of the smart phone. Of note, the slug 120 may be an integral aspect of the armband.

For example, a jogger often wears an armband in which a cell phone may be placed during jogging activities. The slug 120 may be placed in any position in the armband, as long as it is exposed to both the skin and the mobile phone's imaging 112a and lighting system 112b, such as simultaneously or independently. Of course, the skilled artisan may appreciate that other bodily worn devices may be used as the vehicle for the presently disclosed system, such as a smart watch, such as wherein the slug 120 may be exposed to the skin and then placed in front of the smart watch camera, or wherein the smart watch includes an imaging system on the back thereof, such that the slug 120, also on the back of the smart watch, is exposed to the skin and the camera of the smart watch simultaneously.

More particularly, whether the slug 120 is integrated with the armband or similar vehicle, or with a smart phone or smart phone case, the interface 110 may be firmly lodged against the camera and light sources. This adjacency may occur so as to maximize the area of the sensor strip 102 exposed to the mobile device's lighting 112b, and to allow the imaging system's field of view to scan the entire breath of the sensor strip 102.

Of note, the slug interface 110 may depend on customer needs. For example, the slug 120 may be integrated into an exercise device, such as the armband mentioned throughout, which may simultaneously receive the mobile device 112, or the slug 120 may have an interface wholly separate from the mobile device 112, such as wherein the slug is placed into a slot of a case or cover in which the mobile device 112 may be placed, to allow for exposure of the slug 120 to the mobile device 112 via interface 110.

The slug, as referenced herein, may include therein a passive one of sensor strip 102 in order to receive bodily fluids. The slug 120 and/or strip 102 may be single use or multiuse, such as wherein a multiuse slug 120 may experience performance decay, and thus necessitate replacement after a predetermined time period. Different slugs 120 may include different sensor strips 102 particularly suitable to sense different elements, such as a sensor strip for sensing sweat during exercise as differentiated from a sensor strip for sensing saliva.

Needless to say, additional hardware may be associated with the slug 120, such as to improve the analytics provided by the smart phone. For example, an array of light emitting diodes (LEDs) may be included in the slug to better modulate the light to which the sensor strip 102 is exposed, and/or secondary lenses, such as a series of collimating lenses, may be included in the slug 120 in order to improve the acquired images generated by the mobile device's imaging system 112a. By way of example, these integrated hardware elements may be automated as to the slug 120, or may be manipulated by the mobile device 112 once the slug 120 is brought into contact.

The mobile device 112 may include image-processing based analytics specifically to analyze characteristics of the sensor strip 102, such as to analyze with particularity color, hue, and the like in order to provide corresponding output parameters correlated to the bodily fluid, such as the pH, the concentration of ammonia, and the like. As such, the embodiments may include an analytics engine 140 on-board the mobile device's processing system, which may include various algorithms to convert sensed data to instructive data for the user, such as using the correlations indicated in Tables 1 and 2, above. For example, the analytics engine may convert a hue of the sensor strip 102 to a concentration of ammonia in parts per million that may then be displayed to the user. Further, the analytics engine 140 may further provide to the user an indication of what such a level of ammonia might mean specifically to the user.

Moreover, the analytics engine 140 on the mobile device 112 may allow the user to activate and/or modulate aspects or secondary aspects of the disclosed system, such as modulating the light source 112b and imager 112a in order to record a color change of a pH sensor 102. That is, the user may be enabled to manipulate the imaging system 112a and analytics based on that which is to be sensed pursuant to the user's instruction, or such variations may occur automatically.

By way of non-limiting example, the slug 120 may integrate secondary aspects 144 in the form of a series of LEDs with varying emission bands to be used as light sources to sense different characteristics of various slugs 120 having varying sensors 102. Such an LED array 144 be integrated with the mobile device 112, or may be actuatable by the mobile device 112, and may offer flexibility in emission wavelength, such as from the UV to the infrared (IR) range, such as with variable spectral widths, i.e., variable emission band widths.

Thereby, a well-defined emission band for the light sources (112b and/or 144) may provide selectivity for a particular sensor strip 102 in measuring, for example, reflectance. The degree of overlap between absorption bands for certain electrolytes, and the emission bands of the light sources (112b and/or 144), determines the selectivity of the sensor system. Thus, the aforementioned parameters correlated to bodily fluids may be assumed to be constant, and are accordingly defined by their imaging response to particular enhanced LED lighting.

Further, pH indicators may include dyes on the sensor strip, such as bromocresol purple, aniline blue and/or methyl red, which may elicit a colorimetric response in the form of a change in wavelength (in nm) resultant from changes in pH for a range consistent with sweat (pH 4.0 to 8.0). Thus, the percent observance (i.e., the inverse of reflection) of light passing through the sweat may be used as a quantitative marker. That is, light intensity may be detected as a function of time (i.e., weaker versus stronger). More particularly, Table 3 illustrates optical features and variables of an imaging system according to exemplary ones of the embodiments.

TABLE 3

| Sample | LED features | Camera | Image Properties |
|---|---|---|---|
| Mesh size | Pulse width, 100 | Resolution, 5312 × | Lens focal length, |
| ~10-20 mm | ms | 2988 pixels | 28 mm |
| | Max pulse | Video, 1080p @ | Viewing angels, |
| | current, 30 A | 60 fps | 30″, ±45° |
| | Pulse energy, | 2.0 MP | Field of view, |
| | ~2.0-5.0 mJ | (1920 × 1080) | 25 × 25 mm² |
| | Pulse separation, | Acquisition rate, 1 | |
| | ~3 s | Hz | |

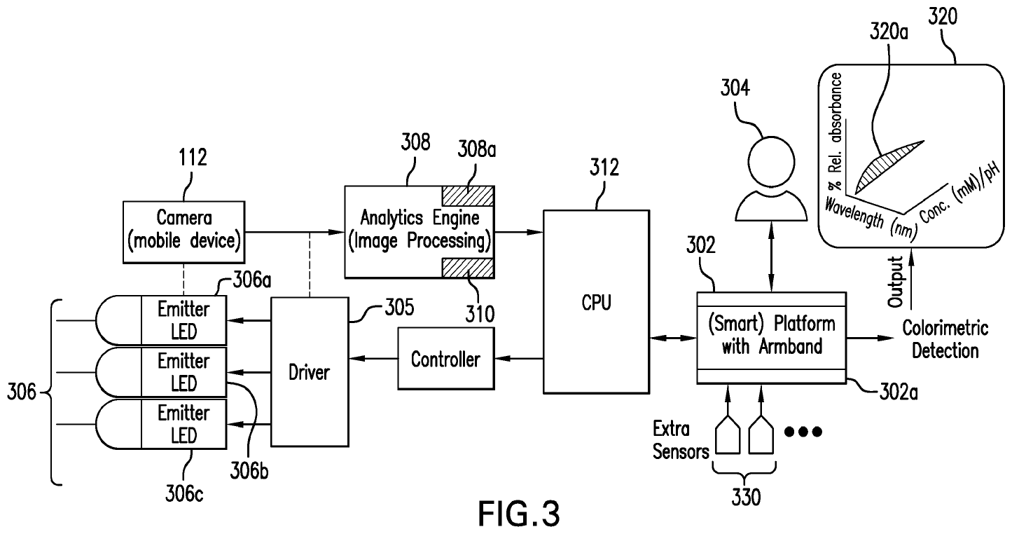

FIG. 3 is a schematic block diagram illustrating the input and colorimetric detection of sweat accrued on a sensor strip 302, such as for pH and/or ammonia detection. Upon input from a user 304 of a mobile device, a driver 305 powers the LED lighting 306 of the mobile device. Of note, an array of LEDs (e.g., red, green, and IR) 306a, 306b, 306c . . . on the mobile device (or as secondary lighting) may interrogate the sweat medium 302a for electrolyte concentration buildup in real time. Acquired data may then be processed through the analytics engine 308, incorporating the image processing 310 pipeline associated with the mobile device's CPU 312.

That is, the imaging system may use the analytics engine 308 associated with the processing system to correlate imaged data to a look-up table 308a within the analytics engine 308 in order to relay to the user 304, upon display of the data, the meaning of the chemical makeup indicated by the sensed data. It will be understood that the look-up tables 308a may be comparable, in simplistic form, to the “databases” illustrated by Tables 1 and 2, above. By way of comparison, a user may use a color-coded strip in one's swimming pool in order to test the pH level, wherein the pH level is indicative of the chemical balance of the pool, and wherein instructions are included to the user as to how to treat the pool based on the pH readings.

The data, once processed, may be output for display in numeric or graphical format 320a on the display 320 of the mobile device, by way of non-limiting example, along with a construction of any meaning of the displayed data. For example, the output 320a may be a plot of electrolyte concentration versus relative absorbance. It will be understood that additional sensors 330 may be included, such as for monitoring skin temperature, heart rate, humidity, and the like, and such secondary sensors 330 may be integrated into an armband or other vehicle in which the slug/sensor strip and/or mobile device reside in order to provide supplementary health data.

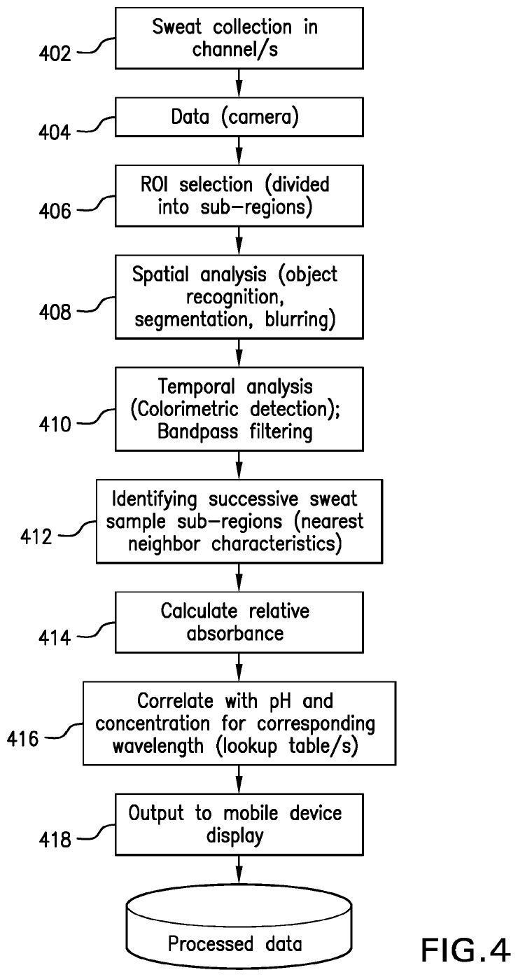

FIG. 4 is a flow chart illustrating an algorithmic analysis method 400 in accordance with the exemplary embodiments. Initially, sweat may be accrued at step 402, and exposed to a mobile camera at step 404. The recorded image data may be filtered and pre-processed at step 406. A region of interest for imaging analysis may be selected and subdivided into an array of pixels at step 406, by way of non-limiting example.

The pre-processed region of interest may then be subjected to processing such as spatial analysis encompassing object recognition, segmentation, and blurring, at step 408. Thereafter, temporal analysis may be undertaken, such as in which colorimetric detection and bandpass filtering are performed, at step 410. Next, the identification of successive sub regions may be undertaken, such as based on nearest neighbor characteristics, at step 412. This may be followed by a computation of the relative absorbance (percentage) at step 414, and a correlation with pH and concentration for a corresponding wavelength at step 416. A numeric or graphical display over time of this data may be then output to a user at step 418.

Sensors used to sense any of various bodily fluids may necessitate insertion or association with various body parts. For example, sensor strips may be provided in a slug to go into the user's nose, in the user's ear canal, or in the user's mouth, such as in order to sense mucus, earwax, or saliva. Likewise, and by way of non-limiting example, slugs may be enabled to sense tears, urine, or feces, and may receive bodily fluids from, for example, armpits, chest, or back. Moreover, the slugs may incur fluids online or off, and may be associated with a smartphone when on line directly or indirectly, i.e., the association may be when the slug is eventually communicative with a consumer electronic device which is, itself, communicative with a smartphone or like device.

In any of the foregoing instances, the slug may be associated with the imaging system of a mobile device, such as by insertion into a single use or multiuse slot, such that the processing system of the mobile device may, through a look up table, provide the user with information on electrolyte balance, insulin balance, the presence of certain medical conditions, and so on. For example, sugar and glucose levels may be sensed in the embodiments, and the slug made may provide this information to the image processing system for analysis, and may indicate whether levels are of concern for any reason.

In any of the foregoing circumstances, an automated or manual identification of the usage of each different strip may be provided to the imager of the mobile device. Thereby, different single use, or one or more multiuse, slugs may indicate to one or more applications 160 on the mobile device to engage in an analysis from the image processing system based on the type of reactive strip in the slug under analysis at that time. Thereby, testing of nearly any bodily fluid may occur in real time using the image processing and processing systems, such as an assessment of cortisone levels for users under duress. For example, a user may be under duress while operating a machine or flying a jet, and the machine or the jet may provide the necessary imaging system and processing system to test each slug.

Further, to the extent the slug is comprised of only simple passive strips, color strips of controlled colors may be printed by users, such as on standard computer printers. Needless to say, such strips would be single use; however, the embodiments do envision multiuse sensor strips. In a multiuse context, a slug may be washable, such as in order to allow for repeated use, particularly dependent on the bodily fluid being sensed by the sensor strip of the slug. In single use or multiuse cases, the sensor strip and slug may preferably be reactive and passive.

Accordingly, the embodiments provide distinct advantages over the known art, such as a low hardware overhead due to the utilization of existing device hardware, i.e., that which is in the customer's own smart phone, for data acquisition and processing. As such, the solution presently provided may be “consumer grade” i.e., although the solution may provide stand-alone hardware, it may leverage existing devices, such as mobile phone devices. This leveraging may include a leveraging of mobile device phone power, lighting, imaging systems, processing capabilities, and the like.

Further, a dedicated software app may incorporate various algorithms for image processing, such as may be unique for particular sensor strips, bodily fluids, conditions, or the like, as discussed herein throughout. Therefore, various use cases are available in the embodiments, such as routine and longitudinal evaluation of bio markers, such as pH and ammonia, as well as measurements of hydration, athletic performance, clinical diagnoses, and remote monitoring based on network conductivity and data upload, by way of non-limiting example.

Needless to say, and in accordance with the foregoing embodiments, custom mobile devices, such as wearable mobile devices, such as armbands or wristbands, may be provided with the disclosed platform embedded therein. Such wearable devices may include the imaging and processing systems disclosed herein, and may be uniquely enabled to identify particular biomarkers, and/or to monitor specific levels of certain chemicals within the body. In such circumstances, a dedicated device may serve via known means to communicate this information to a care provider, such as to allow for monitoring of chronic diseases.

Existing solutions typically entail desktop analytics systems, off-line monitoring, such as by medical personnel, and the like. Further, such known solutions may typically necessitate the use of an off-line swabbing or other manner of providing bodily fluids off-line, or the use of patches, for example. These known solutions are fraught with difficulties, such as inaccuracies due to the requirement of human interpretation and intervention, such as to assess color change, as well as inconvenience to the user, such as due to the need to wear an uncomfortable patch or engage in a swabbing of an area of the body. Yet further, such solutions are inartful, at least in that certain such solutions require dedicated hardware, separate power sources, lack of, or need for secondary wireless conductivity, and may require excessive user interaction. These disadvantages of the known art are cured by the foregoing embodiments.

In the foregoing Detailed Description, it can be seen that various features are grouped together in a single embodiment for the purpose of streamlining the disclosure. This method of disclosure is not to be interpreted as reflecting an intention that the embodiments require more features than are expressly recited herein. That is, the recited embodiments are provided by way of example only, and the disclosure encompasses any embodiments having more or fewer elements than the exemplary embodiments which will be apparent to the skilled artisan in light of the discussion herein.

What is claimed is:

1. A system for correlating bodily fluids to health aspects, comprising:
a slug comprising a passive chemical sensor, a receiving channel for wicking the bodily fluids from a body to the passive chemical sensor, and an interface for interfacing the passive chemical sensor to a mobile device;
at least one indicator associated with the passive chemical sensor, wherein the at least one indicator changes based on features of the wicked bodily fluids; and
at least one computing memory device associated with the mobile device comprising at least comparative lookup of an application, wherein the at least one indicator is compared to the comparative lookup to produce a user display on the application of the health aspects indicated by the at least one indicator.

2. The system of claim 1, wherein the passive chemical sensor comprises a sensor strip.

3. The system of claim 1, wherein the interface to the mobile device comprises an interface to an imaging and lighting system of the mobile device.

4. The system of claim 1, wherein the slug is suitable for placement into a slot in an exercise device.

5. The system of claim 4, wherein the exercise device further comprises a holder for the mobile device.

6. The system of claim 4, wherein the exercise device comprises an armband.

7. The system of claim 5, wherein the holder comprises a pocket.

8. The system of claim 1, wherein the slug further comprises a secondary lighting source.

9. The system of claim 8, wherein the secondary lighting source comprises a LED array.

10. The system of claim 1, wherein the application further comprises an algorithmic analytics platform for particular ones of the bodily fluids.

11. The system of claim 1, wherein the features consist of one of pH and ammonia.

12. The system of claim 1, further comprising at least one secondary sensor.

13. The system of claim 1, wherein the secondary sensor consists of at least one of a hydration sensor and a heartrate sensor.

14. The system of claim 1, wherein the slug is suitable for placement into a slot of the mobile device.

15. The system of claim 1, wherein the slug is suitable for placement into a slot of a protective case associated with the mobile device.

16. The system of claim 1, wherein the passive chemical sensor is single-use.

17. The system of claim 1, wherein the at least one indicator is a color or hue.

18. The system of claim 1, wherein the bodily fluid is one of sweat and saliva.

19. The system of claim 1, wherein the slug further comprises secondary lensing.

20. The system of claim 19, wherein the secondary lensing comprises collimating lenses.

21. The system of claim 1, wherein the health aspects comprise at least one of exercise impact and presence of a health condition.
